(19) United States
(12) Patent Application Publication
(10) Pub. No.: US 2003/0077584 A1
(43) Pub. Date: Apr. 24, 2003

(54) Methods and compositons for bi-directional polymorphism detection

(76) Inventors: Mark Kunkel, Lawrence, NJ (US); Craig Gelfand, Jackson, NJ (US)

Correspondence Address: HOFFMAN & BARON, L.L.P, 350 JERICHO TURNPIKE, JERICHO, NY, 11753-1317, US

(21) Appl. No.: 09/941,138

(22) Filed: Aug. 28, 2001

(51) Int. Cl. C12Q 1/68; C12P 19/34

(52) U.S. Cl. 435/6; 435/91.2

(57) ABSTRACT

The present invention provides methods and compositions for detecting polymorphic sites by employing bi-directional primer extension reactions. In one embodiment, the present invention provides methods and compositions that minimize cost of reagents, such as labeled nucleotides, and minimize the cost of detection instrumentation.

### BACKGROUND OF THE INVENTION

Extensive progress in the field of biotechnology over the last two decades has given rise to new and promising routes to the identification and investigation of diseases. Specifically, advances in nucleic acid synthesis and sequencing have led to the development of the science of genomics. High-throughput sequencing technologies have enabled significant milestones, including the mapping of the human genome. With the ability to rapidly sequence large amounts of DNA, large-scale analysis of genomic characteristics has become possible. Technologies are now evolving to identify and characterize features of the human genome pertinent to individual or population-based variations in genotypes that may be used to identify an individual's susceptibility to a given disease. Among the most promising of avenues for detecting genomic variance in individuals and populations is the analysis and characterization of genetic polymorphisms.

Polymorphisms relate to variances in genomes among different species, for example, or among members of a species, among populations or sub-populations within a species, or among individuals in a species. Such variances are expressed as differences in nucleotide sequences at particular loci in the genomes in question. These differences include, for example, deletions, additions or insertions, rearrangements, or substitutions of nucleotides or groups of nucleotides in a genome.

One important type of polymorphism is a single nucleotide polymorphism (SNP). Single nucleotide polymorphisms occur with a frequency of about 1 in 1,000 base pairs, where a single nucleotide base in the DNA sequence varies among individuals. SNPs may occur both inside and outside the coding regions of genes. It is believed that many diseases, including cancer, hypertension, heart disease, and diabetes, for example, are the result of mutations borne as SNPs or collections of SNPs in subsets of the human population. Currently, one focus of genomics is the identification and characterization of SNPs and groups of SNPs and how they relate to phenotypic characteristics of medical and/or pharmacogenetic relevance, for example.

A variety of approaches to determining, or scoring, the large variety of polymorphisms in genomes have developed. Although these methods are applicable to many types of genomic polymorphisms, they are particularly amenable to determining, or scoring SNPs.

One preferred method of polymorphism detection employs enzyme-assisted primer extension. SNP-IT™ (disclosed by Goelet, P. et al. WO92/15712, and U.S. Pat. Nos. 5,888,819 and 6,004,744, each herein incorporated by reference in its entirety) is a preferred method for determining the identity of a nucleotide at a predetermined polymorphic site in a target nucleic acid sequence. Thus, it is uniquely suited for SNP scoring, although it also has general applicability for determination of a wide variety of polymorphisms. SNP-IT™ is a method of polymorphic site interrogation in which the nucleotide sequence information surrounding a polymorphic site in a target nucleic acid sequence is used to design a primer that is complementary to a region immediately adjacent to the target polynucleotide, but not including the variable nucleotide(s) in the polymorphic site of the target polynucleotide. The primer is extended by a single labeled terminator nucleotide, such as a dideoxynucleotide, using a polymerase, often in the presence of one or more chain terminating nucleoside triphosphate precursors (or suitable analogs). A detectable signal or moiety, covalently attached to the SNP-IT™ primer, is thereby produced. The detectable signal, or moiety, may be attached to the primer either before or after the primer extension reaction.

In some embodiments of SNP-IT™, the oligonucleotide primer is bound to a solid support prior to the extension reaction. In other embodiments, the extension reaction is performed in solution and the extended product is subsequently bound to a solid support. In an alternate embodiment of SNP-IT™, the primer is detectably labeled and the extended terminator nucleotide is modified so as to enable the extended primer product to be bound to a solid support.

Ligase/polymerase mediated genetic bit analysis (U.S. Pat. Nos. 5,679,524, and 5,952,174, both herein incorporated by reference) is another example of a suitable polymerase-mediated primer extension method for determining the identity of a nucleotide at a polymorphic site. Ligase/polymerase SNP-IT™ utilizes two primers. Generally, one primer is detectably labeled, while the other is designed to be bound to a solid support. In alternate embodiments of ligase/polymerase SNP-IT™, the extended nucleotide is detectably labeled. The primers in ligase/polymerase SNP-IT™ are designed to hybridize to each side of a polymorphic site on the same strand, such that there is a gap comprising the polymorphic site. Only a successful extension reaction, followed by a successful ligation reactions enables production of a detectable signal. The method offers the advantages of producing a signal with considerably lower background than is possible by methods employing only hybridization or primer extension alone.

An alternate method for determining the identity of a nucleotide at a predetermined polymorphic site in a target polynucleotide is described in Söderlund et al., U.S. Pat. No. 6,013,431 (the entire disclosure of which is herein incorporated by reference). In this alternate method, nucleotide sequence information surrounding a polymorphic site in a target nucleic acid sequence is used to design a primer that is complementary to a region flanking, but not including the variable nucleotide(s) at the polymorphic site of the target. In some embodiments of this method, following isolation, the target polynucleotide may be amplified by any suitable means prior to hybridization to the interrogating primer. The primer is extended, using a polymerase, often in the presence of a mixture of at least one labeled deoxynucleotide and one or more chain terminating nucleoside triphosphate precursors (or suitable analogs). A detectable signal is produced upon incorporation of the labeled deoxynucleotide into the primer.

The cost of identifying, or genotyping, SNPs ranges from about twenty cents to one dollar or more per DNA sample. Due to the large size of many studies that use SNP information, and expense of SNP analysis, SNP detection must be rapid, amenable to high-throughput, available at low cost, and reliable. One significant cost associated with these assays is the cost of the detectable label, be it a labeled nucleotide or labeled oligonucleotides used in the allele-determination or allele-discrimination process. Labeled nucleotides employed in polymorphism analysis include, for example, chemiluminescent, fluorescent, radioactive, immunoaffinity, and various dye labels. Label detection may be enzyme-assisted, such as, for example, employment of enzyme-linked immunosorbent assay (ELISA) technology in SNPstream 25K®, or immunoaffinity assisted, such as employing indirect fluorescent labeling of haptens with fluorophores. Use of multiple different detectable labels in a single interrogation run is costly. Further, the use of differentially labeled nucleotides generally necessitates the purchase of a bi- or multi-channel detection device. These devices are generally more costly than single-channel detection devices. Sometimes the detection of more than one type of label can be difficult as a result of cross signaling from two or more labels.

Moreover, certain existing detection platforms are inherently limited to single-channel detection. For example, the widely used Luminex LabMAP® system is limited to a single assay result readout channel, having only a single laser and a single photomultiplier to image assay output. In the LabMAP® platform, a single biotinylated nucleotide is labeled after a reaction run with a fluorophore for detection by flow cytometry. Currently, a single tagged SNP-IT™ primer is used to genotype any particular polymorphic locus. Thus, two otherwise identical reactions must be run with biotin labeling of each alternative allelic terminating nucleotide, in order to generate a single genotype. The Luminex platform is an example where the single-channel limitation is more from the perspective of cost reduction of the instrument, since multi-channel read-outs, for example, using multiple lasers and photomultipliers, can be envisioned within current technology limits.

Other detection technologies are more limited to single-channel read-outs because of the physical nature of the platforms themselves. One example is the BioStar® platform. This detection system employs a unique thin-film preparation of a silicon surface that can be reacted for highly sensitive assay readout. As currently available, a single ELISA step is used to generate a signal. Signal detection, however, is achieved through a change of mass on the surface rather than by color per se, although a color change is a simple method of imaging the mass change. This platform relies on a mass change and does not inherently allow for multi-color detection. As a result, two separate reactions must be carried out to generate one genotype if a single primer is used.

Because most SNPs are bi-allelic, multiple runs must be carried out if employing single channel detection systems, or multiple costly labeled nucleotides must be employed with multi-channel systems, in order to interrogate a single biallelic SNP. Due to the cost of labeled nucleotides and the prevalence of single-channel detection systems, it is desirable to develop new approaches to detecting SNPs as well as other polymorphisms. Methods and compositions that minimize cost of reagents, such as labeled nucleotides, and minimize the cost of detection instrumentation, are highly desireable. Further, methods and compositions that allow high throughout multiplex detection of polymorphisms would be beneficial.

### SUMMARY OF THE INVENTION

The present invention provides methods and compositions that minimize cost of reagents, such as labeled nucleotides, and minimize the cost of detection instrumentation. Further, the present invention provides methods and compositions that allow high throughput multiplex detection of polymorphisms.

In one embodiment, the present invention provides a method for identifying one or more nucleotides present at a polymorphic site on one or more alleles comprising the steps of: a) obtaining an upper strand of target nucleic acids from the one or more alleles and a lower strand of target nucleic acids from the one or more alleles, wherein each strand comprises a polymorphic site; b) hybridizing an upper strand primer that is complementary to the upper strand of target nucleic acids at a region immediately adjacent to the polymorphic site on the upper strand of target nucleic acids so as to obtain one or more unpaired nucleotide bases to be identified on the upper strand, and hybridizing a lower strand primer that is complementary to the lower strand of target nucleic acids at a region immediately adjacent to the polymorphic site on the lower strand so as to obtain one or more unpaired nucleotide bases to be identified on the lower strand; c) exposing the hybridized upper and lower strand primers to a polymerization agent in a mixture comprising one or more nucleotides so that one or more primer extension products are formed if the one or more nucleotides in the mixture is complementary to the polymorphic site on the upper strand or lower strand of target nucleic acids; and d) separating any one or more primer extension products from unextended primers so as to identify the polymorphic site on the one or more alleles.

In a second embodiment, the present invention provides a method for identifying one or more nucleotides present at a polymorphic site on one or more alleles comprising the steps of: a) obtaining an upper strand of target nucleic acids from the one or more alleles and a lower strand of target nucleic acids from the one or more alleles; wherein each strand comprises the polymorpic site; b) hybridizing an upper strand primer that is complementary to the upper strand of target nucleic acids at a region immediately adjacent to the polymorphic site on the upper strand of target nucleic acids so as to obtain one or more unpaired nucleotide bases to be identified on the upper strand, and hybridizing a lower strand primer that is complementary to the lower strand of target nucleic acids at a region immediately adjacent to the polymorphic site on the lower strand so as to obtain one or more unpaired nucleotide bases to be identified on the lower strand; the upper and lower strand primers each have a unique tag at the 5′ end capable of binding to known positions on a solid support; c) exposing the hybridized upper and lower strand primers to a polymerization agent in a mixture comprising one or more nucleotides so that one or more primer extension products are formed if the one or more nucleotides in the mixture is complementary to the polymorphic site on the upper strand or lower strand of target nucleic acids; d) contacting the solid support with the mixture so as to cause each unique sequence tag to bind to known positions on the solid support; and e) detecting each bound primer, wherein the positions of the primers on the solid support in conjunction with any one or more primer extension products allows identification of the polymorphic site on the one or more alleles.

In a third embodiment, the present invention provides a method for identifying one or more nucleotides present at a polymorphic site on the one or more alleles comprising the steps of: a) obtaining an upper strand of target nucleic acids from the one or more alleles and a lower strand of target nucleic acids from the one or more alleles, wherein each strand comprises the polymorphic site; b) hybridizing an upper strand primer that is complementary to the upper strand of target nucleic acids at a region immediately adjacent to the polymorphic site on the upper strand of target nucleic acids so as to obtain an unpaired nucleotide base to be identified at the polymorphic site on the upper strand, and hybridizing a lower strand primer that is complementary to the lower strand of target nucleic acids at a region immediately adjacent to the polymorphic site on the lower strand so as to obtain an unpaired nucleotide base to be identified at the polymorphic site on the lower strand; wherein the upper and lower primers each have a unique tag at the 5′ end capable of binding to known positions on a solid support; and c) exposing the hybridized upper and lower strand primers to a polymerization agent in a mixture comprising at least four different terminating nucleotides so as to form primer extension products wherein the primers are extended bidirectionally when the terminating nucleotide in the mixture is complementary to the polymorphic site on the upper strand or lower strand of target nucleic acids; wherein at least two different terminating nucleotides have the same detectable characteristic; d) contacting the solid support with the mixture so as to cause each unique sequence tag to bind to known positions on the solid support; and e) detecting each bound primer, wherein the positions of the primers on the solid support in conjunction with any detectable characteristic allows identification of the polymorphic site on the one or more alleles.

In a fourth embodiment, the present invention provides a method for identifying one or more nucleotides present at a polymorphic site on one or more alleles comprising the steps of: a) obtaining an upper strand of target nucleic acids from the one or more alleles and a lower strand of target nucleic acids from the one or more alleles, wherein each strand comprises the polymorphic site; b) hybridizing an upper strand primer that is complementary to the upper strand of target nucleic acids at a region immediately adjacent to the polymorphic site on the upper strand of target nucleic acids so as to obtain one or more unpaired nucleotide bases to be identified on the upper strand, and hybridizing a lower strand primer that is complementary to the lower strand of target nucleic acids at a region immediately adjacent to the polymorphic site on the lower strand so as to obtain one or more unpaired nucleotide bases to be identified on the lower strand; c) exposing the hybridized upper and lower strand primers to a polymerization agent in a mixture comprising one or more nucleotides so that one or more primer extension products are formed if the one or more nucleotides in the mixture are complementary to the polymorphic site on the upper strand or lower strand of target nucleic acids wherein the primers are extended bidirectionally; and d) separating any one or more primer extension products from unextended primers so as to identify the polymorphic site on the one or more alleles.

For a better understanding of the present invention together with other and further advantages and embodiments, reference is made to the following description taken in conjunction with the examples, the scope of which is set forth in the appended claims.

### BRIEF DESCRIPTION OF THE FIGURES

Preferred embodiments of the invention have been chosen for purposes of illustration and description, but are not intended in any way to restrict the scope of the invention. The preferred embodiments of certain aspects of the invention are shown in the accompanying figures, wherein:

FIG. 1 illustrates one embodiment of the invention. A sample biallelic DNA comprising an A/G allele is mixed with primers that are complementary to the upper and lower strands of the allele, where the 3′-end of the primers end immediately adjacent to the polymorphic nucleotide. The primers bear a unique tag that allows for discrimination between the upper and lower strand primers. Two labeled nucleotides and a polymerase are added. Single nucleotide primer extension occurs in a bi-directional fashion. That is, primers are extended by a single labeled nucleotide on the upper and the lower strands. The primers bearing the labeled nucleotides are exposed to an addressable array by, for example, hybridizing to their immobilized complement, and a single channel detection system identifies the label on upper and/or lower strand primers. Because the array is addressable, the location of signal on the array will identify a labeled primer as an upper or lower strand primer, thereby revealing the nucleotide at the polymorphic site on each allele. (Primer extension with nucleotides not bearing a label has not been shown for simplicity).

FIG. 2 illustrates another embodiment of the invention. A sample biallelic DNA containing an A/T allele is mixed with primers that are complementary to the upper and lower strands of the allele, where the 3′-end of the primers end immediately adjacent to the polymorphic nucleotide. The primers have a unique tag that allows for discrimination between them. Only one labeled nucleotide, a labeled A or T, here, a labeled A, and a polymerase are added. Single nucleotide primer extension occurs in a bi-directional fashion. That is, primers are extended by a single labeled nucleic acid on the upper and lower strands. The primers bearing the labeled nucleotide are applied to an addressable array by, for example, hybridizing to the immobilized complement, and a single channel detection system identifies the label on upper and/or lower strand primers. Because the array is addressable, the location of the signal on the array will identify a labeled primer as an upper or lower strand primer, thereby revealing the nucleotide at the polymorphic site on each allele. (Primer extension with nucleotides not bearing a label has not been shown for simplicity).

FIG. 3 illustrates one of the G/C loci genotyped using a single labeled terminator (G-TAMRA) and three unlabeled terminators. There are three distinct clusters that represent the genotypes—the cluster on the left, with P value about 0.0, denotes a CC homozygote group, the cluster in the middle, P value about 0.5, shows the heterozygote GC group, and the group on the right, P value about 1.0, is the homozygote GG group. The Y axis represents a sum of all detected allele signal intensities, and is used as a confidence measure, for distinguishing actual signal from background. Shown in the graph are the genotypes for 24 central samples genotypes against one locus, SNP 1230 from Orchid's SNP database.

FIG. 4 illustrates a failed SNP scoring run using single-color bi-directional SNP-IT™ due to a functional failure of one of the two bi-directional SNP-IT™ primers.

FIGS. 5 and 6 illustrate Locus 1451 genotyped separately with each SNP-IT™ primer using a two-color SNP-IT™ assay. It is evident that the upper SNP-IT™ primer failed, but the lower SNP-IT™ primer yields accurate genotypes.

FIG. 7 and 8 illustrate two-color bi-directional SNP-IT™ assays that make it possible to genotype all four bases in one well. In the example shown, a G/C SNP is typed in each direction with G and C labeled differently, thus each bi-directional SNP-IT™ primer contains corroborating 2-color genotyping data, adding higher confidence to the genotyping results because of this redundancy. By labeling all four bases with one of only two labels, it is possible to genotype all four bases. In such an experiment, the only requirement is that G and C not bear the same label, and that A and T not bear the same labels.

FIG. 9 illustrates the bi-directional two color genotypes obtained from use of a GenFlex® chip from Affymetrix, Inc., using the upper strand SNP-IT™ primer pool. The genotypes obtained for the different samples are 100% concordant across the two platforms. Genotyping failures are identical across the two platforms further confirming SNP-IT™ primer design problems, that result in failed assays in a reproducible fashion.

### DETAILED DESCRIPTION OF THE INVENTION

The present invention provides methods and compositions that minimize cost of reagents, such as labeled nucleotides, and minimize the cost of detection instrumentation. Further, the present invention provides methods and compositions that allow high throughout multiplex detection of polymorphisms.

Target Nucleic Acids

The present invention includes obtaining a target nucleic acid sequence encompassing a known polymorphism. The target nucleic acid sequence will preferably be “biologically active” with regard to the capacity of this nucleic acid to hybridize to another oligonucleotide or polynucleotide molecule. Target nucleic acid sequences may be either DNA or RNA, single-stranded or double-stranded or a DNA/RNA hybrid duplex. The target nucleic acid sequence may be a polynucleotide or oligonucleotide. Preferred target nucleic acid sequences are between 40 to about 200 nucleotides in length, in order to facilitate detection. The target nucleic acid sequence can be cut or fragmented into these segments by methods known in the art e.g., by mechanical or hydrodynamic shearing methods such as sonication, or by enzymatic methods such as restriction enzymes or nucleases.

The target nucleic acid may be isolated, or derived from a biological sample. The term “isolated” as used herein refers to the state of being substantially free of other material such as non nuclear proteins, lipids, carbohydrates, or other materials such as cellular debris or growth media with which the target nucleic acid may be associated. Typically, the term “isolated” is not intended to refer to a complete absence of these materials. Neither is the term “isolated” generally intended to refer to the absence of stabilizing agents such as water, buffers, or salts, unless they are present in amounts that substantially interfere with the methods of the present invention. The term “sample” as used herein generally refers to any material containing nucleic acid, either DNA or RNA or DNA/RNA hybrids. These samples can be from any source including plants and animals. Generally, such material will be in the form of a blood sample, a tissue sample, cells directly from individuals or propagated in culture, plants, yeast, fungi, mycoplasma, viruses, archaebacteria, histology sections, or buccal swabs, either fresh, fixed, frozen, or embedded in paraffin or another fixative.

Preferably, the target nucleic acids are from genomic DNA drawn from a diverse population of humans so as to do genetic mapping or haplotyping or other studies. Such genomic DNA contains polymorphic site(s) and is used to amplify a region encompassing the polymorphic site(s) of interest through an amplification method such as, for example, the Polymerase Chain Reaction (PCR). Typically the PCR reaction is multiplexed, where 10 to 12 or more polymorphic sequences are amplified simultaneously in the same reaction vessel. These polymorphisms are pooled together so as to obtain for example, SNPs having desirable characteristics. For example, in one embodiment, target nucleic acids containing SNPs bearing the same two polymorphic alleles are combined.

The target nucleic acid may be single-stranded and may be derived from either the upper or lower strand nucleic acids of double stranded DNA, RNA or other nucleic acid molecules. The upper strand of target nucleic acids includes the plus strand or sense strand of nucleic acids. The lower strand of target nucleic acids is intended to mean the minus or antisense strand that is complementary to the upper strand of target nucleic acids. Thus, reference may be made to either strand and still comprise the polymorphic site and a primer may be designed to hybridize to either or both strands. Target nucleic acids are not meant to be limited to sequences within the coding regions, but may also include any region of a genome or portion of a genome containing at least one polymorphism.

Polymorphisms

The target nucleic acid sequences or fragments thereof contain the polymorphic site(s), or includes such site(s) and sequences located either distal or proximal to the sites(s). These polymorphic sites or mutations may be in the form of deletions, insertions, re-arrangement, repetitive sequence, base modifications, or base changes at a particular site in a nucleic acid sequence. This altered sequence and the more prevalent, or normal, sequence may co-exist in a population. In some instances, these changes confer neither an advantage nor a disadvantage to the species or individuals within the species, and multiple alleles of the sequence may be in stable or quasi-stable equilibrium. In some instances, however, these sequence changes will confer a survival or evolutionary advantage to the species, and accordingly, the altered allele may eventually over time be incorporated into the genome of many or most members of that species. In other instances, the altered sequence confers a disadvantage to the species, as where the mutation causes or predisposes an individual to a genetic disease or defect. As used herein, the terms “mutation” or “polymorphic site” refers to a variation in the nucleic acid sequence between some members of a species, a population within a species or between species. Such mutations or polymorphisms include, but are not limited to, single nucleotide polymorphisms (SNPs), one or more base deletions, or one or more base insertions.

Polymorphisms may be either heterozygous or homozygous within an individual. Homozygous individuals have identical alleles at one or more corresponding loci on homologous chromosomes. Heterozygous individuals have different alleles at one or more corresponding loci on homologous chromosomes. As used herein, alleles include an alternative form of a gene or nucleic acid sequence, either inside or outside the coding region of a gene, including introns, exons, and untranscribed or untranslated regions. Alleles of a specific gene generally occupy the same location on homologous chromosomes. A polymorphism is thus said to be “allelic,” in that, due to the existence of the polymorphism, some members of a species carry a gene with one sequence (e.g., the original or wild-type “allele”), whereas other members may have an altered sequence (e.g., the variant or, mutant “allele”). In the simplest case, only one mutated variant of the sequence may exist, and the polymorphism is said to be biallelic. For example, if the two alleles at a locus are indistinguishable (for example A/A), then the individual is said to be homozygous at the locus under consideration. If the two alleles at a locus are distinguishable (for example A/G), then the individual is said to be heterozygous at the locus under consideration. The vast majority of known single nucleotide polymorphisms are bi-allelic—where there are two alternative bases at the particular locus under consideration.

Primers

The present invention utilizes one or more upper and lower strand primers. In order for an oligonucleotide to serve as a primer, it typically need only be sufficiently complementary in sequence to be capable of forming a double-stranded structure under the conditions employed. Establishing such conditions typically involves selection of solvent and salt concentration, incubation temperatures, incubation times, assay reagents and stabilization factors. The term “primer” or “primer oligonucleotide” refers to an oligonucleotide as defined herein, which is capable of acting as a point of initiation of synthesis when employed under conditions in which synthesis of a primer extension product that is complementary to a nucleic acid strand is induced, as, for example, in a DNA replication reaction such as a PCR reaction. Like non-primer oligonucleotides, primer oligonucleotides may be labeled according to any technique known in the art, such as with radioactive atoms, fluorescent, enzymatic labels, proteins, haptens, antibodies, sequence tags, and the like.

Primers can be polynucleotides or oligonucleotides capable of being extended in a primer extenson reaction at their 3′ end. As used herein, the term “polynucleotide” includes nucleotide polymers of any number. The term “oligonucleotide” includes a polynucleotide molecule comprising any number of nucleotides, preferably, less than about 200 nucleotides. More preferably, oligonucleotides are between 5 and 100 nucleotides in length. Most preferably, oligonucleotides are 15 to 45 nucleotides in length. The exact length of a particular oligonucleotide or polynucleotide, however, will depend on many factors, which in turn depend on its ultimate function or use. Short primers generally require lower temperatures to form sufficiently stable hybrid complexes with a template. The primers of the present invention should be complementary to the upper or lower strand target nucleic acids. Preferably, the primers should not have self complementarity involving their 3′ end' in order to avoid primer fold back leading to self-priming architectures and assay noise. Preferred primers of the present invention include oligonucleotides from about 8 to about 40 nucleotides in length, to longer polynucleotides that may be up to several thousand nucleotides long.

Primers of about 10 nucleotides are the shortest sequence that can be used to selectively hybridize to a complementary target nucleic acid sequence against the background of non-target nucleic acids in the present state of the art. Most preferably, sequences of at least 20 to about 25 nucleotides are used to assure a sufficient level of hybridization specificity.

The primers of this invention must be capable of specifically hybridizing to the target nucleic acid sequence—such as, for example, one or more upper primers hybridizing to one or more upper strand target nucleic acids. Likewise, one or more lower primers must be capable of hybridizing to one or more lower strand target nucleic acids. As used herein, two nucleic acid sequences are said to be capable of specifically hybridizing to one another if the two molecules are capable of forming an anti-parallel, double-stranded nucleic acid structure or hybrid under conditions sufficient to promote such hybridization, whereas they must be substantially unable to form a double-stranded structure or hybrid when incubated with a non-target nucleic acid sequence under the same conditions. A nucleic acid molecule is said to be the “complement” of another nucleic acid molecule if it exhibits complete sequence complementarity. As used herein, molecules are said to exhibit “complete complementarity” when every nucleotide of one of the molecules is able to form a base pair with a nucleotide of the other. Two molecules are said to be “substantially complementary” if they can hybridize to one another with sufficient stability to permit them to remain annealed to one another under at least conventional low-stringency conditions. Similarly, the molecules are said to be “complementary” if they can hybridize to one another with sufficient stability to permit them to remain annealed to one another under conventional high-stringency conditions. Conventional stringency conditions are described, for example, in Sambrook, J., et al., in Molecular Cloning, a Laboratory Manual, 2nd Edition, Cold Spring Harbor Press, Cold Spring Harbor, N.Y. (1989), and by Haymes, B. D., et al. in Nucleic Acid Hybridization, A Practical Approach, IRL Press, Washington, D.C. (1985), both herein incorporated by reference). Departures from complete complementarity are therefore permissible, as long as such departures do not completely preclude the capacity of the molecules to form a double-stranded structure or hybrid.

The primers of the present invention optionally may be tagged at the 5′ end. Tags include any label such as radioactive labels, fluorescent, enzymatic labels, proteins, haptens, antibodies, sequence tags, and the like. Preferably, the tag does not interfere with the processes of the present invention. Typically, a tag may be attached to the 5′ end of the primer, with the remainder of the primer sequence being complementary to the target strand. The most preferred tag includes unique tags or marking each type of primer with a distinct sequence that is complementary to a sequence bound to a solid support, where such solid support may include an array, including an addressable array. Thus, when the primer is exposed to the solid support under suitable hybridization conditions, the tag hybridizes with the complementary sequence bound to the solid support. In this way, the identity of the primer can be determined by geometric location on the array, or by other means of identifying the point of association of the tag with the probe. For example, upper strand primers have a unique 5′ tag to differentiate them from lower strand primers with respect to the particular polymorphic site. Sequences complementary to the 5′ tag are bound to the solid support at discrete positions (for example, upper strand, lower strand positions) on, for example, an addressable array.

Alternatively, tags can be non-complementary bases, or longer sequences that can be interspersed into the primer provided that the primer sequence has sufficient complementarity with the sequence of the target strand to hybridize therewith for the purposes employed. However, for detection purposes, the primers in the most preferred embodiment should have exact complementarity to obtain the optimal results. Thus, primers employed in the present invention must generally be complementary in sequence and be able to form a double-stranded structure or hybrid with a target nucleotide sequence under the particular conditions employed.

Primer Extension

One preferred method of detecting polymorphic sites employs enzyme-assisted primer extension. SNP-IT™ (disclosed by Goelet, P. et al. WO92/15712, and U.S. Pat. Nos. 5,888,819 and 6,004,744, each herein incorporated by reference in its entirety) is a preferred method for determining the identity of a nucleotide at a predetermined polymorphic site in a target nucleic acid sequence. Thus, it is uniquely suited for SNP scoring, although it also has general applicability for determination of a wide variety of polymorphisms. SNP-IT™ is a method of polymorphic site interrogation in which the nucleotide sequence information surrounding a polymorphic site in a target nucleic acid sequence is used to design an oligonucleotide primer that is complementary to a region immediately adjacent to at the 3′ or 5′ end of the target polynucleotide, but not including the variable nucleotide(s) in the polymorphic site of the target polynucleotide. The target polynucleotide is isolated from a biological sample and hybridized to the interrogating primer. Following isolation, the target polynucleotide may be amplified by any suitable means prior to hybridization to the interrogating primer. The primer is extended by a single labeled terminator nucleotide, such as a dideoxynucleotide, using a polymerase, often in the presence of one or more chain terminating nucleoside triphosphate precursors (or suitable analogs). A detectable signal is thereby produced. As used herein, immediately adjacent to the polymorphic site includes from about 1 to about 100 nucleotides, more preferably from about 1 to about 25 nucleotides in the 3′ or 5′ direction of the polymorphic site. Most preferably, the primer is hybridized one nucleotide immediately adjacent to the polymorphic site in either the 3′ or 5′ direction.

In some embodiments of SNP-IT™, the primer is bound to a solid support prior to the extension reaction. In other embodiments, the extension reaction is performed in solution (such as in a test tube or a micro well) and the extended product is subsequently bound to a solid support. In an alternate embodiment of SNP-IT™, the primer is detectably labeled and the extended terminator nucleotide is modified so as to enable the extended primer product to be bound to a solid support. An example of this includes where the primer is fluorescently labeled and the terminator nucleotide is a biotin-labeled terminator nucleotide and the solid support is coated or derivatized with avidin or streptavidin. In such embodiments, an extended primer would thus be capable of binding to a solid support and non-extended primers would be unable to bind to the support, thereby producing a detectable signal dependent upon a successful extension reaction.

Ligase/polymerase mediated genetic bit analysis (U.S. Pat. Nos. 5,679,524, and 5,952,174, both herein incorporated by reference) is another example of a suitable polymerase mediated primer extension method for determining the identity of a nucleotide at a polymorphic site. Ligase/polymerase SNP-IT™ utilizes two primers. Generally, one primer is detectably labeled, while the other is designed to be affixed to a solid support. In alternate embodiments of ligase/polymerase SNP-IT™, the extended nucleotide is detectably labeled. The primers in ligase/polymerase SNP-IT™ are designed to hybridize to each side of a polymorphic site, such that there is a gap comprising the polymorphic site. Only a successful extension reaction, followed by a successful ligation reaction enables production of the detectable signal. The method offers the advantages of producing a signal with considerably lower background than is possible by methods employing either hybridization or primer extension alone.

An alternate method for determining the identity of a nucleotide at a polymorphic site in a target polynucleotide is described in Söderlund et al., U.S. Pat. No. 6,013,431 (the entire disclosure is herein incorporated by reference). In this method, the nucleotide sequence surrounding a polymorphic site in a target nucleic acid sequence is used to design an oligonucleotide primer that is complementary to a region flanking the 3′ or 5′ end of the target polynucleotide, but not including the variable nucleotide(s) in the polymorphic site of the target polynucleotide. The target polynucleotide is isolated from the biological sample and hybridized with an interrogating primer. In some embodiments of this method, following isolation, the target polynucleotide may be amplified by any suitable means prior to hybridization with the interrogating primer. The primer is extended, using a polymerase, often in the presence of a mixture of at least one labeled deoxynucleotide and one or more chain terminating nucleoside triphosphate precursors (or suitable analogs). A detectable signal is produced on the primer upon incorporation of the labeled deoxynucleotide into the primer.

The primer extension reaction of the present invention employs a mixture of one or more labeled nucleotides and a polymerizing agent. The term “nucleotide” or nucleic acid as used herein is intended to refer to ribonucleotides, deoxyribonucleotides, acylic derivatives of nucleotides, and functional equivalents or derivatives thereof, of any phosphorylation state capable of being added to a primer by a polymerizing agent. Functional equivalents of nucleotides are those that act as substrates for a polymerase as, for example, in an amplification method. Functional equivalents of nucleotides are also those that may be formed into a polynucleotide that retains the ability to hybridize in a sequence-specific manner to a target polynucleotide. Examples of nucleotides include chain-terminating nucleotides, most preferably dideoxynucleoside triphosphates (ddNTPs), such as ddATP, ddCTP, ddGTP, and ddTTP; however other terminators known to those skilled in the art, such as acyclonucleotide analogs or arabinoside triphosphates, are also within the scope of the present invention. These ddNTPs differ from conventional 3′ deoxynucleoside triphosphates (dNTPs) in that they lack a hydroxyl group at the 3′ position of the sugar component.

Preferred polymerizing agents include polymerases. Preferred polymerases for performing single base extensions using the methods and apparatus of the invention are polymerases exhibiting little or no exonuclease activity. More preferred are polymerases that tolerate and are active at temperatures greater than physiological temperatures, for example, at 50° C. to 70° C. or are tolerant of temperatures of at least 90° C. to about 95° C. Preferred polymerases include Taq® polymerase from T. aquaticus (commercially available from Perkin-Elmer Cetus, Foster City, Calif.), Sequenase® and ThermoSequenase® (commercially available from U.S. Biochemical, Cleveland, Ohio), and Exo(-) polymerase (commercially available from New England Biolabs, Beverley, Mass.).

The primer extension reaction of the present invention can employ one or more labeled nucleotide bases. Preferably, two, three or four nucleotides of different bases. Depending on the polymorphic site being interrogated, if one type of nucleotide is being used in the primer reaction mixture, some primers will not be extended. If four different nucleotides are used, both upper and lower strand primers may be extended.

The nucleotides employed may bear a detectable characteristic. As used herein a detectable characteristic includes any identifiable characteristic that enables distinction between nucleotides. It is important that the detectable characteristic does not interfere with any of the methods of the present invention. Detectable characteristic refers to an atom or molecule or portion of a molecule that is capable of being detected employing an appropriate method of detection. Detectable characteristics include inherent mass, electric charge, electron spin, mass tag, radioactive isotope, dye, bioluminescent molecule, chemiluminescent molecule, nucleic acid molecule, hapten molcule, protein molecule, light scattering/phase shifting molecule, or fluorescence molecules. As used herein, the phrase “same detectable characteristic” includes nucleotides that are detectable because they have the same signal. The same detectable characteristic includes embodiments where nucleotides are labeled with the same type of labels, for example, A and C nucleotide may be labeled with the same type of dye, where they emit the same type of signal.

Nucleotides and primers may be labeled according to any technique known in the art. Preferred labels include radiolabels, fluorescent labels, enzymatic labels, proteins, haptens, antibodies, sequence tags, mass tags, fluorescent tags and the like. Preferred dye type labels include, but are not limited to, TAMRA (carboxy-tetramethylrhodamine), ROX (carboxy-X-rhodamine), FAM (5-carboxyfluorescein), and the like.

In the most preferred embodiment one or two different type of nucleotides are labeled with the same type of label. For example, (A) nucleotides are labeled with TAMRA and (C) nucleotides are labeled with TAMRA.

Bidirectional Primer Extension

The term “bi-directional” or bi-directionally refers to primer extension occurring in an anti-parallel fashion with respect to the upper and lower primers. For example, primer extension may occur at the 3′ end of the primer for one or more upper and lower primers. However for the one or more upper strand primers, extension may occur right to left. In contrast, for one or more lower strand primers, extension may occur left to right, but still in the 5′ to 3′ direction. Thus, primer extension may occur in an anti-parallel or bi-directional fashion. Preferably, this bi-directional primer extension is done substantially simultaneously in one reaction well. Accordingly, the method of the present invention is adaptable for multiplex, high throughput genotyping of one or more alleles.

In one embodiment of the present invention, one or more upper strand primers having a nucleotide sequence complementary to the upper strand target sequence is hybridized immediately adjacent to the polymorphic site of interest to form a duplex so as to obtain one or more unpaired nucleotide bases to be identified on the upper strand. Similarly, one or more lower strand primers having a nucleotide sequence complementary to the lower strand target sequence is hybridized immediately adjacent to the polymorphic site of interest to form a duplex so as to obtain one or more unpaired nucleotide bases to be identified on the lower strand. As a result, when one or more labeled nucleotides are added, depending upon the particular allele(s) being interrogated, primer extension will proceed on both the upper and the lower strands substantially simultaneously. This situation results in primer extension proceeding in a bi-directional manner; that is to say, primer extension on the upper strand will proceed in the right to left direction, whereas primer extension on the lower strand will proceed in the left to right direction, but both primers may be extended in the 5′ to 3′ direction.

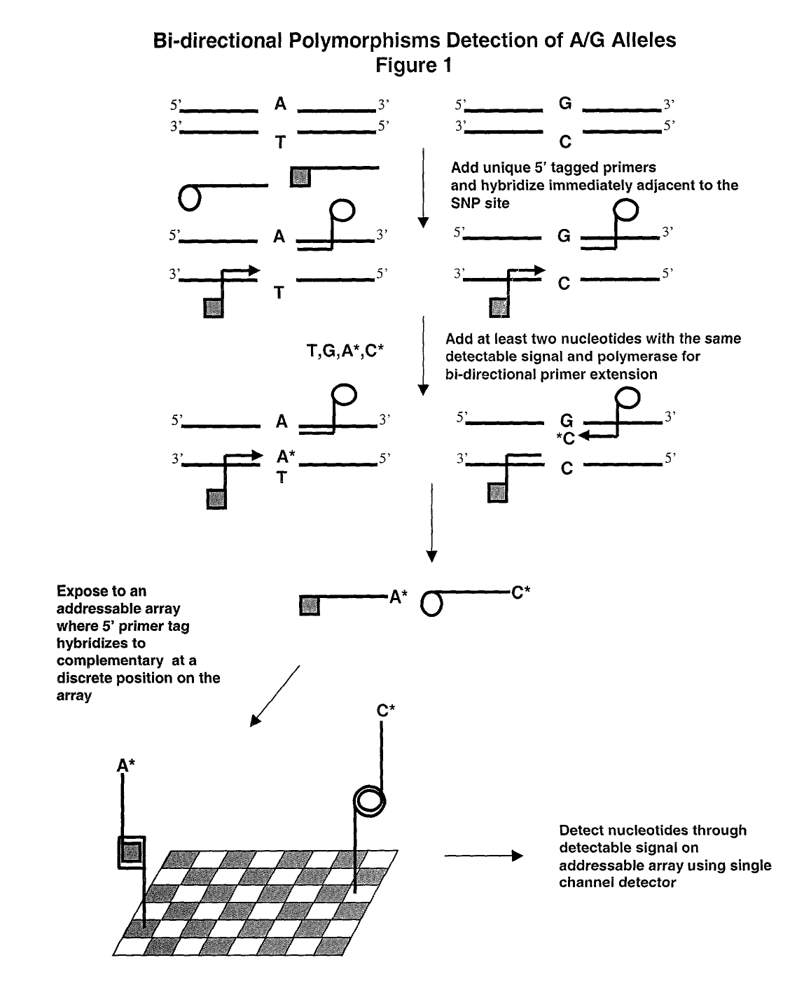

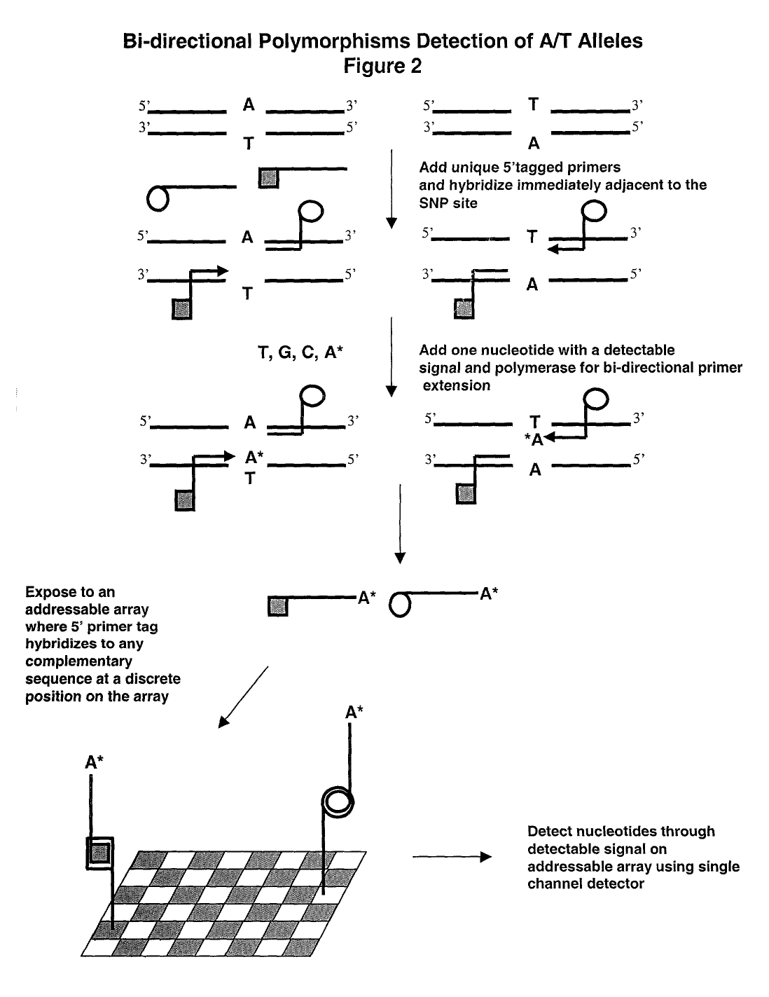

FIG. 1 illustrates one example of bi-directional SNP detection. For the case of interrogating a biallelic A/G polymorphism, employing four or more nucleotides, where two labeled nucleotides carry the same label. Another embodiment of the present invention is also illustrated in FIG. 2, for the case of interrogating a biallelic A/T polymorphism, employing a single labeled nucleotide. Allele determination using this single color method is achieved in part through employing primer tags on the primers for the upper and lower strands, as indicated in the embodiment illustrated in FIGS. 1 and 2. Such tags may include the inherent sequence of the primer itself. The bi-directional SNP detection method of the present invention in one embodiment, employs both upper and lower strand primers, one or more labeled nucleotides, and a single color label that can be detected by a single channel detection device. Primer separation is based upon unique primer tag features that allows for the economical determination of polymorphic site.

Advantages of the bi-directional single color reaction scheme of this invention, over the standard multi-color reaction scheme, are illustrated in Table A. Label requirements for the six possible biallelic polymorphisms are provided.

TABLE A

| | | LABELED NUCLEOTIDES FOR POLY- | |
|---|---|---|---|
| | | MORPHISM DETECTION REACTIONS | |
| | | STANDARD | BI-DIRECTIONAL |
| | | MULTI-COLOR | SINGLE COLOR |
| | | REACTION | REACTION |
| ALLELES | A/G | A* & G** | A* & C* |
| INTERROGATED | T/C | T* & C** | T* & G* |
| | A/C | A* & C** | A* & G* |
| | T/G | T* & G** | T* & C* |
| | A/T | A* & T** | A* or T* |
| | G/C | G* & C** | G* or C* |

*Detectable signal of one color
**Detectable signal of another color

Table A shows that the standard multi-color protocol requires the use of labeled nucleotides bearing different detectable signals, whereas the bi-directional single color scheme allows for one kind of detectable signal to be employed on any labeled nucleotides used in the assay. It is advantageous to employ nucleotides with only one kind of detectable characteristic in that it allows detection by a single channel detection device. Such devices are generally more economical than multi-channel detection devices. Further, different types of detectable characteristics may lead to difficulties in interpreting the results due to mixed signals. Moreover, certain existing systems, such as the BioStar and Luminex systems employed in the art of biochemical analyses, are single channel systems. Also, Table A also reveals that for two biallelic polymorphisms, A/T and G/C, only a single labeled nucleotide is required to successfully interrogate those alleles. This effectively reduces the cost of interrogating those alleles in half, because the majority of the cost of carrying out an interrogation reaction is associated with the cost of the labeled nucleotide.

In addition, the inherent two-fold information coming from both strands of DNA means that any particular polymorphism can be typed in one of two different schemes. For instance, a SNP locus defined as having A and G alternative allees could just as easily be described as having T and C alternative alleles on the opposite strand. Further, the definition of “upper” and “lower” strand target nucleic acids is used for references purposes, such that allele definition is inextricably linked to the “sidedness” of the surrounding sequence. The result of this two-fold information content is that each listing in the table above can have a complementary allelic content counterpart if the sidedness of the polymorphism is reversed. The genetic content, and the results of genotyping assays, remains unchanged.

Separation and Detection

Once the bi-directional primer extension reaction is employed, extended and unextended primers (if any) can be separated from each other so as to identify the polymorphic site on the one or more alleles that are interrogated. Separation of nucleic acids can be performed by any methods known in the art. Some separation methods include the detection of DNA duplexes with intercalating dyes such as, for example, ethidium bromide, hydbridization methods to detect specific sequences and/or separate or capture oligonucleotide molecules whose structures are known or unknown and hybridization methods in connection with blotting methods well known in the art. Hybridization methods may be combined with other separation technologies well known in the art, such as separation of tagged oligonucleotides through solid phase capture, such as, for example, capture of hapten-linked oligonucleotides to immunoaffinity beads, which in turn may bear magnetic properties. Solid phase capture technologies also includes DNA affinity chromatography, wherein an oligonucleotide is captured by an immobilized oligonucleotide bearing a complementary sequence. Specific polynucleotide tails may be engineered into oligonucleotide primers, and separated by hybridization with immobilized complementary sequences. Such solid phase capture technologies also includes capture onto streptavidin-coated beads (magnetic or nonmagnetic) of biotinylated oligonucleotides. DNA may also be separated and with more traditional methods such as centrifugation, electrophoretic methods or precipitation or surface deposition methods. This is particularly so when the extended or unextended primers are in solution phase. The term “solution phase” is used herein to refer to a homogenous or heterogenous mixture. Such a mixture may be aqueous, organic, or contain both aqueous and organic components. As used herein, the term “solution” should be construed to be synonymous with suspension in that it should be construed to include particles suspended in a liquid medium.

The polymorphic sites can be detected by any means known in the art. One method of detection of nucleotides is by fluorescent techniques. Fluorescent hybridization probes may be constructed that are quenched in the absence of hybridization to target nucleic acid sequences. Other methods capitalize on energy transfer effects between fluorophores with overlapping absorption and emission spectra, such that signals are detected when two fluorophores are in close proximity to one another, as when captured or hybridized.

Nucleotides may also be detected by, or labeled with moieties that can be detected by, a variety of spectroscopic methods relating to the behavior of electromagnetic radiation. These spectroscopic methods include, for example, electron spin resonance, optical activity or rotation spectroscopy such as circular dichroism spectroscopy, fluorescence polarization, absorption/emission spectroscopy, ultraviolet, infrared, or mass spectroscopy, Raman spectroscopy, visible spectroscopy, and nuclear magnetic resonance spectroscopy.

The term “detection” refers to identification of a detectable moiety or moieties. The term is intended to include the ability to identify a moiety by electromagnetic characteristics, such as, for example, charge, light, fluorescence, chemiluminescense, changes in electromagnetic characteristics such as, for example, fluorescence polarization, light polarization, dichroism, light scattering, changes in refractive index, reflection, infrared, ultraviolet, and visible spectra, and all manner of detection technologies dependent upon electromagnetic radiation or changes in electromagnetic radiation. The term is also intended to include identification of a moiety based on binding affinity, intrinsic mass, mass deposition, and electrostatic properties.

Single channel detection refers to instrumentation or methods limited to simultaneous or non-simultaneous detection of a single characteristic of a detectable moiety or moieties. Bi-channel detection refers to instrumentation or methods of simultaneous or non-simultaneous detection of a characteristic of a detectable moiety or moieties. Multiple-channel detection refers to instrumentation or methods limited to simultaneous or non-simultaneous detection of or more characteristic of a detectable moiety or moieties.

One single channel platforms suitable for use with the present invention is the Luminex LabMAP system which is limited to a single assay result readout channel, having only a single laser and a single photomultiplier to image assay output. In the LabMAP platform, a single biotinylated nucleotide is labeled after a reaction run with a fluorophore for detection by flow cytometry.

Another single channel detection system suitable for use with the present invention is the BioStar platform. This detection system employs a unique thin-film preparation of a silicon surface that can be reacted for highly sensitive assay readout. As currently available, a single ELISA step with a precipitating is used to generate a signal. Signal detection, however, is achieved through a change of mass on the surface rather than by color per se, although a color change is a simple method of imaging the mass change.

Another method of detecting the nucleotide present at the polymorphic site is by comparison of the concentrations of free, unincorporated nucleotides remaining in the reaction mixture at any point after the primer extension reaction. Mass spectroscopy in general and, for example, electrospray mass spectroscopy, may be employed for the detection of unincorporated nucleotides in this embodiment. This detection method is possible because only the nucleotide(s) complementary to the polymorphic base is (are) depleted in the reaction mixture during the primer extension reaction. Thus, mass spectrometry may be employed to compare the relative intensities of the mass peaks for the nucleotides, Likewise, the concentrations of unlabeled primers may be determined and the information employed to arrive at the identity of the nucleotide present at the polymorphic site.

Solid Support

Preferred separation methods employ exposing any extended and unextended primers to a solid support. Solid supports include arrays. The term “array” is used herein to refer to an ordered arrangement of immobilized biological molecules at a plurality of positions on a solid, semi-solid, gel or polymer phase. This definition includes phases treated or coated with silica, silane, silicon, silicates and derivatives thereof, plastics and derivatives thereof such as, for example, polystyrene, nylon and, in particular, polystyrene plates, glasses and derivatives thereof, including derivatized glass, glass beads, controlled pore glass (CPG). Immobilized biological molecules includes oligonucleotides that may include other moieties, such as tags and/or affinity moieties. The term “array” is intended to include and be synonymous with the terms “chip,” “biochip,” “biochip array,” “DNA chip,” “RNA chip,” “nucleotide chip,” and “oligonucleotide chip.” All these terms are intended to include arrays of arrays, and are intended to include arrays of biological polymers such as, for example, oligonucleotides and DNA molecules whose sequences are known or whose sequences are not known.

Preferred arrays for the present invention include, but are not limited to, addressable arrays including an array as defined above wherein individual positions have known coordinates such that a signal at a given position on an array may be identified as having a particular identifiable characteristic. The terms “chip,” “biochip,” “biochip array,” “DNA chip,” “RNA chip,” “nucleotide chip,” and “oligonucleotide chip,” are intended to include combinations of arrays and microarrays. These terms are also intended to include arrays in any shape or configuration, 2-dimensional arrays, and 3-dimensional arrays.

One particularly preferred array is the GenFlex™ Tag Array, from Affymetrix, Inc., that is comprised of capture probes for 2000 tag sequences. These are 20 mers selected from all possible 20 mers to have similar hybridization characteristics and at least minimal homology to sequences in the public databases.

Another preferred array is the addressable array that has reverse complements to the unique 5′ tags of the upper and lower primers. These reverse complements are bound to the array at known positions. This type of tag hybridizes with the array under suitable hybridization conditions. By locating the bound primer in conjunction with detecting one or more extended primers, the nucleotide identity at the polymorphic site can be determined.

In one preferred embodiment of the present invention, the target nucleic acid sequences are arranged in a format that allows multiple simultaneous detections (multiplexing), as well as parallel processing using oligonucleotide arrays.

In another embodiment, the present invention includes virtual arrays where extended and unextended primers are separated on an array where the array comprises a suspension of microspheres, where the microspheres bear one or more capture moieties to separate the uniquely tagged primers. The microspheres, in turn, bear unique identifying characteristics such that they are capable of being separated on the basis of that characteristic, such as for example, diameter, density, size, color, and the like.

Compositions

The present invention provides genotyping, haplotyping, and diagnostic compositions including kits that have upper and lower primers for bi-directional primer extension. The kit may also include one or more containers, as well as additional reagent(s) and/or active and/or inert ingredient(s) for performing any variations on the methods of the invention. Exemplary reagents include, without limitation, at least two or more primers, one or more terminator nucleotides, such as dideoxynucleotides, that are labeled with a detectable marker, and one or more polymerases. The kits can also include instructions for mixing or combining ingredients or use.

Having now generally described the invention, the same may be more readily understood through the following reference to the following examples, which are provided by way of illustration and are not intended to limit the present invention unless specified.

### EXAMPLES

The examples below illustrate bi-directional primer extension and polymorphism identification of the present invention. Both alleles of DNA are genotyped at a SNP site using the same label, despite the fact that the SNPs are biallelic. For example, in the case of a G/C or an A/T SNP, a labeled G terminator and a labeled A terminator would be used. But in the event that the SNP is A/C, T/G, A/G, or T/C, both complementary terminating bases are utilized in the extension reaction but they are labeled with the same moiety, for example fluorescein. This feature enables SNP-IT™ technology to be used on single wavelength or single-channel read-out instruments and platforms, while allowing the user to genotype both alleles. By using two of the non-complementary bases, for example, labeled G and A terminators, all four bases can be genotyped.

### Example 1

Materials and Methods: Thermalcycler, multiplexed PCR Primers, 2×hybridization solution, dNTP mix, unlabeled nucleotide terminators, labeled nucleotide terminators, 100 mM Tris-HCl, pH 8.3, 500 mM KCl, 25 mM MgCl₂, exonuclease I and Shrimp Alkaline Phosphatase, Thermosequenase® I, multiplexed upper and lower SNP-IT™ primer pools with each primer in the pool at 100 nM final concentration, glass plate arrayed with 3′ disulfide probes, water, 96-well PCR plates, Eppendorf tubes, Assorted size pipette tips, Wash A—1M SSPE, 0.01% Tween-20,Wash B—0.5M SSPE, and 0.01% Tween-20.

Genomic DNA is used to amplify a region encompassing the SNPs of interest through PCR. Typically the PCR reaction is multiplexed where 10 to 12 SNP sequences are amplified at the same time in the same well. These SNPs are grouped together by extension mix i.e., SNPs having the same two alternative alleles. The PCR reaction is set up as follows:

| Final Concentration | | |
|---|---|---|
| PCR Upper Primer | 50 | nM |
| Lower PCR Primer | 50 | nM |
| dNTPs | 75 | μM each |
| HCl | 50 | mM |
| Tris-HCl, pH 8.3 | 10 | mM |
| MgCl₂ | 5 | mM |

| | |
|---|---|
| Taq Gold ® | 2.5 U/25 ul rxn |
| Genomic DNA | 10 ng/25 ul rxn |

PCR amplification conditions are as follows:

| | |
|---|---|
| Step 1. | 95° C. for 5:00 |
| Step 2. | 95° C. for 0:30 |
| Step 3. | 50° C. for 0:55 |
| Step 4. | 72° C. for 0:30 |
| Step 5. | Go to step 2, 4 times |
| Step 6. | 95° C. for 0:30 |
| Step 7. | 50° C. for 0:55 + 0.2° C. per Cycle |
| Step 8. | 72° C. for 0:30 |
| Step 9. | Go to step 6, 24 times |
| Step 10. | 95° C. for 0:30 |
| Step 11. | 55° C. for 0:55 |
| Step 12. | 72° C. for 0:30 |
| Step 13. | Go to 10, 4 times |
| Step 14. | 72° C. for 7:00 |
| Step 15. | 4° C. hold forever |

Following PCR amplification, the product is cleaned with Exonuclease I and Shrimp Alkaline Phosphatase. Exonuclease I digests away the excess unextended PCR primers and SAP inactives free nucleotide triphosphates remaining from the PCR reaction. The digestion is set up in the thermocycler at 37° C. for 30 min and the enzymes are then heat-inactivated at 95° C. for 10 min.

### Example 2

Single-well, single-color extension is set up using a single labeled nucleotide in the event of a G/C (G) or A/T (A) SNP, and both labeled nucleotides (same label on both) for A/G, T/C, A/C, and T/G SNPs. Extension reactions are set up using Tamra-labeled nucleotides. The SNP-IT™ reaction typically consists of the following.

| | Volume |
|---|---|
| Reagent | in μl |
| Cleaned PCR Product | 12 |
| Tag-SNP IT ™ Primer Pool, upper | 2.5 |
| Tag-SNP IT ™ Primer Pool, lower | 2.5 |
| 1 M Tris HCl, pH 9.5 | 1.7 |
| 100 mM MgCl2 | 2.2 |
| Bodipy Fluorescence nucleotide, 62.5 Um | 0.33 |
| Tamra nucleotide, 62.5 Um | 0.33 |
| Unlabeled nucleotide, 6.25 Um | 0.33 |
| Unlabeled nucleotide, 6.25 Um | 0.33 |
| Thermosequenase ®(32 U/μl) | 0.078 |
| Water 10.702 | |
| Total Volume | 33 |

The extension reactions are carried out using the following thermal cycling method.

Step 1. 96° C. for 3:00 min

Step 2. 94° C. for 0:20 sec

Step 3. 40° C. for 0:11 sec

Step 4. Go to Step 2, 45 times

Step 5. 4° C. hold forever

Following extension, 67 μl of hybridization mix is added to each well of extended product. The hybridization mix contains the following components:

50 μl 2×hybridization solution

15 μl DNAse, RNAse free water

2 μ50×Denhardt's solution

The total volume of the ready-to-hybridize extension reaction is 100 μl. 10 μl of the reaction is added to each well of a micro-arrayed glass plate that has probes arrayed on it, complementary to the tag sequences on the SNP-IT™ primers. Hybridization is carried out at 42° C. for 2 hours under 100% humidity. The hybridized plate is then washed 3× with Wash A and 3× with Wash B and imaged. The SNP-IT™ reactions were also tested on the SNPCode® platform using the Genflex® chips provided by Affymetrix, Inc.

### Example 3

23 DNA samples from the Coriell Cell Repositories were used to genotype against 10 G/C SNPs. These samples are part of a larger group of cell lines combined to represent a diverse human population. Genotyping was done using both single-color and two-color. Using G-Bodipy fluorescein terminator and a C-Tamra terminator accomplishes two-color genotyping. Single color genotyping was done using G-Tamra terminator. In both cases, be it a single-color or two-color SNP-IT™, all four terminators were used with only one or two of the terminators being labeled. All failures seen were due to SNP-IT™ primer design failure on one of the strands. Any primer design issues were confirmed when each of the SNP-IT™ primers was used separately in a SNP-IT™ assay using two labeled terminators. One primer completely failed while the SNP-IT™ primer on the other strand yielded good genotypes. But, for the single-color assay, failure of one of the primers causes genotyping failure on one allele and hence yields a failed SNP, in this case an apparent monoallelic population where polymorphisms are already known to be present. Table 1 represents the genotypes from several of the loci in the multiplexed reaction.

Locus

| Sample | 332 | 386 | 637 | 1039 | 1201 | 1230 |
|---|---|---|---|---|---|---|
| PD02 | CC | CC | GG | CC | CC | GG |
| PD03 | GC | CC | CC | GC | GC | GC |
| PD04 | CC | CC | GC | GC | GC | GG |
| PD05 | GG | CC | GC | CC | CC | CC |
| PD06 | GG | CC | CC | GC | CC | CC |
| PD07 | GC | GC | GC | CC | CC | CC |
| PD08 | GC | CC | GC | CC | CC | CC |
| PD09 | GG | GG | GC | CC | CC | CC |
| PD10 | GG | GC | GC | CC | CC | CC |
| PD11 | CC | CC | CC | CC | GC | GC |
| PD12 | GC | GC | CC | GC | CC | CC |
| PD13 | CC | CC | GG | GC | CC | CC |
| PD14 | CC | CC | GC | CC | CC | GC |
| PD15 | GC | CC | CC | GC | CC | CC |
| PD16 | CC | CC | GG | CC | CC | GC |
| PD17 | CC | GC | GC | CC | CC | GC |
| PD18 | GC | CC | CC | CC | GC | GC |
| PD19 | CC | CC | CC | CC | CC | CC |
| PD20 | GC | GC | GC | CC | CC | GC |
| PD21 | CC | CC | GG | CC | GG | CC |
| PD22 | GG | CC | GC | CC | CC | CC |
| PD23 | GC | GC | GC | CC | CC | CC |
| PD24 | GG | CC | GC | CC | GC | GC |

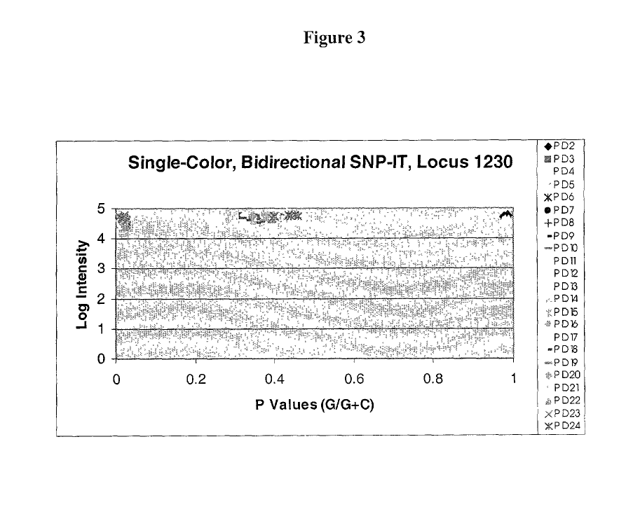

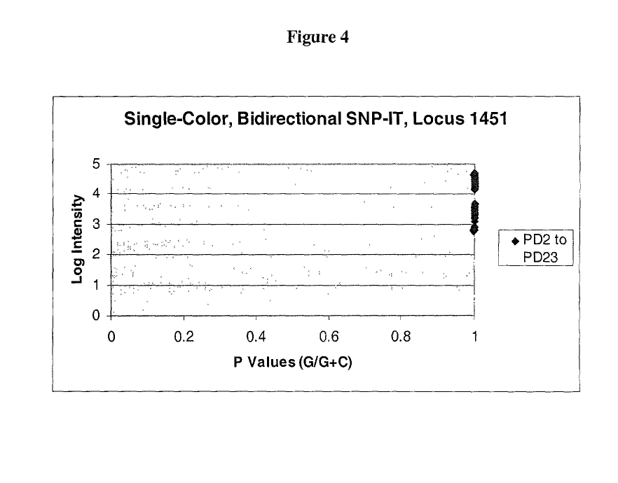

For this assay, the SNP-IT™ primer is tagged with an additional 20-base tag sequence at the 5′ end. This tag sequence is complementary to a probe sequence that is arrayed on the glass plates. Each SNP-IT™ primer is tagged to a unique tag sequence and in a 10-plex single-color, bi-directional SNP-IT ™ reaction, there are a total of 20-tagged SNP-IT™ primers (10 upper strand, and 10 lower strand). There are hence 20 unique probe sequences complementary to the tags arrayed on a glass plate. This probe-tag combination is used to spatially sort SNPs in a multiplexed genotyping reaction. FIG. 3 shows one of the G/C loci genotyped using a single labeled terminator (G-Tamra) and the other three unlabeled terminators. There are three distinct clusters that represent the genotypes—the cluster on the left denotes a CC homozygote group, the cluster in the middle shows the heterozygote GC group, and the group on the right is the homozygote GG group. FIG. 4 shows failed genotyping of locus 1451 using single-color bi-directional SNP-IT™ due to one of the SNP-IT™ primer design failures.

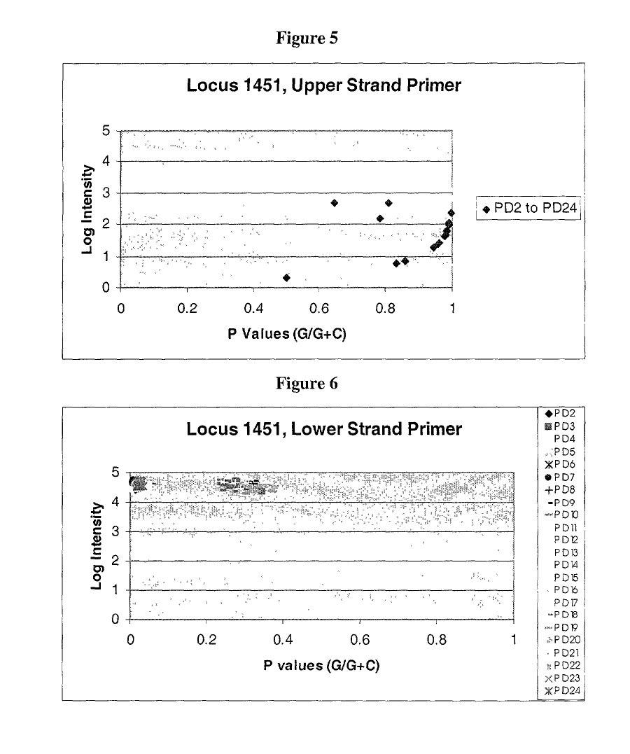

FIGS. 5 and 6 represent Locus 1451 genotyped separately with each SNP-IT™ primer using a two-color SNP-IT™ assay. It is evident that the upper SNP-IT™ primer failed and the lower SNP-IT™ primer yields accurate genotypes. Hence, when the two primers are used in combination for the single-color SNP-IT™ assay, the locus fails as shown in FIG. 4.

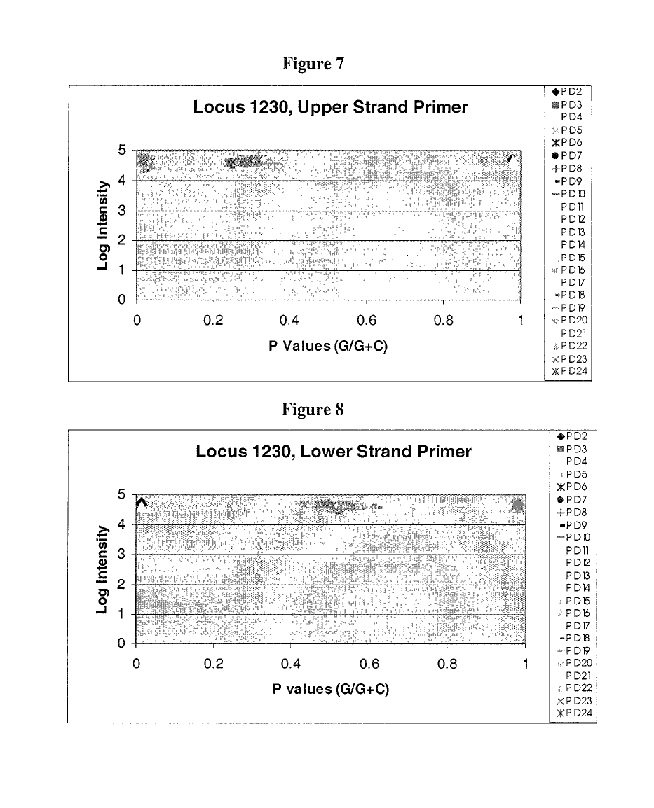

The two-color bi-directional SNP-IT™ assay makes it possible to genotype all four bases in one well. Shown below in FIGS. 7 and 8 are the two-color bi-directional SNP-IT™ results for each of the SNP-IT™ primers. The two-color bi-directional SNP-IT™ assay was also validated on the SNPCode platform that utilizes GenFlex™ chips supplied by Affymetrix, Inc. A spatial sorting mechanism (similar to what is used on the micro arrayed glass plates) is used on the chips as well to sort a multiplexed SNP-IT™ assay.

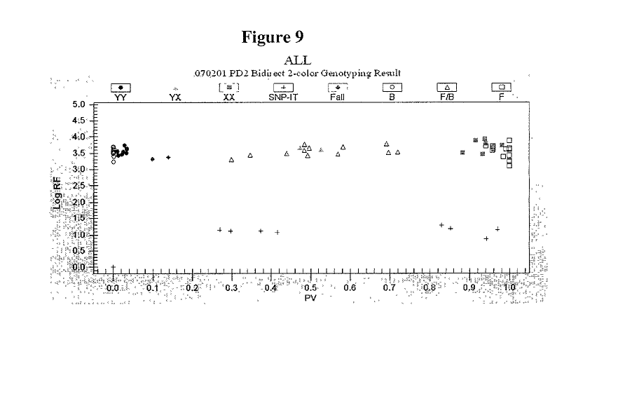

FIG. 9 shows the genotypes obtained from a GenFlex chip using the upper strand SNP-IT™ primer pool. The genotypes obtained for the different samples are 100% concordant across the two platforms. Genotyping failures are identical across the two platforms further confirming SNP-IT™ primer design problems, that result in failed assays in a reproducible fashion.

In conclusion, it can be said that the single-color or two-color bi-directional SNP-IT™ assay is adaptable to different platforms still yielding accurate and reproducible genotypes.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method for identifying one or more nucleotides present at a polymorphic site on one or more alleles comprising the steps of:
a) obtaining an upper strand of target nucleic acids from the one or more alleles and a lower strand of target nucleic acids from the one or more alleles, wherein each strand comprises a polymorphic site;
b) hybridizing an upper strand primer that is complementary to the upper strand of target nucleic acids at a region immediately adjacent to the polymorphic site on the upper strand of target nucleic acids so as to obtain one or more unpaired nucleotide bases to be identified on the upper strand, and hybridizing a lower strand primer that is complementary to the lower strand of target nucleic acids at a region immediately adjacent to the polymorphic site on the lower strand so as to obtain one or more unpaired nucleotide bases to be identified on the lower strand;
c) exposing the hybridized upper and lower strand primers to a polymerization agent in a mixture comprising one or more nucleotides so that one or more primer extension products are formed if the one or more nucleotides in the mixture is complementary to the polymorphic site on the upper strand or lower strand of target nucleic acids; and
d) separating any one or more primer extension products from unextended primers so as to identify the polymorphic site on the one or more alleles.

2. A method according to claim 1, wherein the mixture comprises one or more chain terminating nucleotides.

3. A method according to claim 2, wherein the one or more chain terminating nucleotides with the same detectable characteristic.

4. A method according to claim 2, wherein the one or more chain terminating nucleotides are selected from the group consisting of dideoxynucleotides and acyclonucleotides.

5. A method according to claim 1, wherein the mixture comprises at least four different chain terminating nucleotides.

6. A method according to claim 5, wherein the mixture comprises at least four different chain terminating nucleotides, at least two different chain terminating nucleotides have the same detectable characteristic.

7. A method according to claim 6, wherein at least two different terminating nucleotides are distinguished from each other by a detectable characteristic selected from the group consisting of inherent mass, electric charge, electron spin, mass tag, radioactive isotope, dye, bioluminescent molecule, chemiluminescent molecule, nucleic acid molecule, hapten molcule, protein molecule, light scattering/phase shifting molecule, or fluorescence molecule.

8. A method according to claim 1, wherein the upper and lower strand primers each contain a unique tag at the 5′ end of each primer.

9. A method according to claim 8, wherein each unique tag comprises a sequence that is capable of hybridizing with a complementary sequence at known positions on a solid support.

10. A method according to claim 9, wherein each unique tag comprises from about 8 to about 40 nucleotides.

11. A method according to claim 9, wherein the solid support is selected from the group consisting of silica gel, silicon, glass, polystyrene, nylon, polypropylene, nitrocellulose or CPG.

12. A method according to claim 8, wherein each unique tag comprises a sequence that is capable of hybridizing with a complementary sequence at known positions on an array.

13. A method according to claim 1, wherein the upper and lower strand of target nucleic acids are genomic or mitochondrial DNA.

14. A method according to claim 1, wherein the polymorphic site is selected from the group consisting of a single nucleotide polymorphism, an insertion, a deletion, a re-arrangement, or a repetitive sequence.

15. A method according to claim 1, wherein the method is performed in solution phase in one or more single wells.

16. A method for identifying one or more nucleotides present at a polymorphic site on one or more alleles comprising the steps of:
a) obtaining an upper strand of target nucleic acids from the one or more alleles and a lower strand of target nucleic acids from the one or more alleles; wherein each strand comprises the polymorpic site;
b) hybridizing an upper strand primer that is complementary to the upper strand of target nucleic acids at a region immediately adjacent to the polymorphic site on the upper strand of target nucleic acids so as to obtain one or more unpaired nucleotide bases to be identified on the upper strand, and hybridizing a lower strand primer that is complementary to the lower strand of target nucleic acids at a region immediately adjacent to the polymorphic site on the lower strand so as to obtain one or more unpaired nucleotide bases to be identified on the lower strand; the upper and lower strand primers each have a unique tag at the 5′ end capable of binding to known positions on a solid support;
c) exposing the hybridized upper and lower strand primers to a polymerization agent in a mixture comprising one or more nucleotides so that one or more primer extension products are formed if the one or more nucleotides in the mixture is complementary to the polymorphic site on the upper strand or lower strand of target nucleic acids;
d) contacting the solid support with the mixture so as to cause each unique sequence tag to bind to known positions on the solid support; and
e) detecting each bound primer, wherein the positions of the primers on the solid support in conjunction with any one or more primer extension products allows identification of the polymorphic site on the one or more alleles.

17. A method according to claim 16, wherein the mixture comprises one or more chain terminating nucleotides.

18. A method according to claim 17, wherein the one or more chain terminating nucleotides have the same detectable characteristic.

19. A method according to claim 17, wherein the one or more chain terminating nucleotides are selected from the group consisting of dideoxynucleotides or acyclonucleotides.

20. A method according to claim 16, wherein the mixture comprises at least four different chain terminating nucleotides.

21. A method according to claim 20, wherein the mixture comprises at least four different chain terminating nucleotides, at least two different chain terminating nucleotides have the same detectable characteristic.

22. A method according to claim 21, wherein at least two different terminating nucleotides are distinguishable from each other by a detectable characteristic selected from the group consisting of inherent mass, electric charge, electron spin, mass tag, radioactive isotope, dye, bioluminescent molecule, chemiluminescent molecule, nucleic acid molecule, hapten molecule, protein molecule, light scattering/phase shifting molecule, or fluorescence molecule.

23. A method according to claim 16, wherein each unique tag comprises a nucleic acid sequence that is capable of hybridizing with a complementary sequence at known positions on a solid support.

24. A method according to claim 23, wherein each unique tag comprises from about 8 to about 40 nucleotides.

25. A method according to claim 23, wherein the solid support is selected from the group consisting of silica gel, silicon, glass, polystyrene, nylon, polypropylene, nitrocellulose or CPG.

26. A method according to claim 16, wherein each unique tag comprises a nucleic acid sequence that is capable of hybridizing with a complementary sequence at known positions on an array.

27. A method according to claim 16, wherein the upper and lower strand of target nucleic acids are genomic or mitochondrial DNA.

28. A method according to claim 16, wherein the polymorphic site is a single nucleotide polymorphism, an insertion, a deletion, a re-arrangment, or repetitive sequence.

29. A method according to claim 16, wherein step (c) is performed in solution phase in one or more single wells.

30. A method for identifying one or more nucleotides present at a polymorphic site on the one or more alleles comprising the steps of:
a) obtaining an upper strand of target nucleic acids from the one or more alleles and a lower strand of target nucleic acids from the one or more alleles, wherein each strand comprises the polymorphic site;
b) hybridizing an upper strand primer that is complementary to the upper strand of target nucleic acids at a region immediately adjacent to the polymorphic site on the upper strand of target nucleic acids so as to obtain an unpaired nucleotide base to be identified at the polymorphic site on the upper strand, and hybridizing a lower strand primer that is complementary to the lower strand of target nucleic acids at a region immediately adjacent to the polymorphic site on the lower strand so as to obtain an unpaired nucleotide base to be identified at the polymorphic site on the lower strand; wherein the upper and lower primers each have a unique tag at the 5′ end capable of binding to known positions on a solid support; and
c) exposing the hybridized upper and lower strand primers to a polymerization agent in a mixture comprising at least four different terminating nucleotides so as to form primer extension products wherein the primers are extended bidirectionally when the terminating nucleotide in the mixture is complementary to the polymorphic site on the upper strand or lower strand of target nucleic acids; wherein at least two different terminating nucleotides have the same detectable characteristic;
d) contacting the solid support with the mixture so as to cause each unique sequence tag to bind to known positions on the solid support; and
e) detecting each bound primer, wherein the positions of the primers on the solid support in conjunction with any detectable characteristic allows identification of the polymorphic site on the one or more alleles.

31. A method according to claim 30, wherein the four different terminating nucleotides are selected from the group consisting of dideoxynucleotides or acyclonucleotides.

32. A method according to claim 30, wherein the detectable characteristic is selected from the group consisting of inherent mass, electric charge, electron spin, mass tag, radioactive isotope, dye, bioluminescent molecule, chemiluminescent molecule, nucleic acid molecule, hapten molecule, protein molecule, light scattering/phase shifting molecule or fluorescence molecule.

33. A method according to claim 30, wherein each unique tag comprises a nucleic acid sequence that is capable of hybridizing with a complementary sequence at known positions on a solid support.

34. A method according to claim 30, wherein each unique tag comprises from about 8 to about 40 nucleotides.

35. A method according to claim 30, wherein the solid support is selected from the group consisting of silica gel, silicon, glass, polystyrene, nylon, polypropylene, nitrocellulose or CPG.

36. A method according to claim 30, wherein each unique tag comprises a nucleic acid sequence that is capable of hybridizing with a complementary sequence at known positions on an array.

37. A method according to claim 30, wherein the upper and lower target nucleic acids are genomic or mitochondrial DNA.

38. A method according to claim 30, wherein step (c) is performed in solution phase on one or more single wells.

39. A method for identifying one or more nucleotides present at a polymorphic site on one or more alleles comprising the steps of:
a) obtaining an upper strand of target nucleic acids from the one or more alleles and a lower strand of target nucleic acids from the one or more alleles, wherein each strand comprises the polymorphic site;
b) hybridizing an upper strand primer that is complementary to the upper strand of target nucleic acids at a region immediately adjacent to the polymorphic site on the upper strand of target nucleic acids so as to obtain one or more unpaired nucleotide bases to be identified on the upper strand, and hybridizing a lower strand primer that is complementary to the lower strand of target nucleic acids at a region immediately adjacent to the polymorphic site on the lower strand so as to obtain one or more unpaired nucleotide bases to be identified on the lower strand;
c) exposing the hybridized upper and lower strand primers to a polymerization agent in a mixture comprising one or more nucleotides so that one or more primer extension products are formed if the one or more nucleotides in the mixture are complementary to the polymorphic site on the upper strand or lower strand of target nucleic acids wherein the primers are extended bidirectionally; and
d) separating any one or more primer extension products from unextended primers so as to identify the polymorphic site on the one or more alleles.

40. A method according to claim 5, wherein the mixture comprises at least four different chain terminating nucleotides, at least one chain terminating nucleotide has a label.

41. A method according to claim 20, wherein the mixture comprises at least four different chain terminating nucleotides, at least one chain terminating nucleotide has a label.

42. A method according to claim 1, wherein the upper and lower strand primers are immobilized to a solid support.

43. A method according to claim 1, wherein the upper strand target and lower strand target nucleic acids are amplified prior to hybridization.

44. A method according to claim 1, wherein the method is performed on multiple upper and lower strand targets nucleic acids simultaneously.

45. A method according to claim 1, wherein the method is performed on two alleles.

46. A method according to claim 1, wherein step (e) is performed by single channel detection.

47. A method for identifying one or more nucleotides present at a polymorphic site on one or more alleles comprising the steps of:
a) obtaining an upper strand of target nucleic acids from the one or more alleles and a lower strand of target nucleic acids from the one or more alleles, wherein each strand comprises a polymorphic site;
b) hybridizing an upper strand primer that is complementary to the upper strand of target nucleic acids at a region immediately adjacent to the polymorphic site on the upper strand of target nucleic acids so as to obtain one or more unpaired nucleotide bases to be identified on the upper strand, and hybridizing a lower strand primer that is complementary to the lower strand of target nucleic acids at a region immediately adjacent to the polymorphic site on the lower strand so as to obtain one or more unpaired nucleotide bases to be identified on the lower strand;
c) exposing the hybridized upper and lower strand primers to a polymerization agent in an extension mixture comprising one or more nucleotides so that one or more primer extension products are formed if the one or more nucleotides in the extension mixture is complementary to the polymorphic site on the upper strand or lower strand of target nucleic acids; and
d) detecting in the extension mixture one or more nucleotides not incorporated into the one or more primer extension products so as to identify the one or more nucleotides present at the polymorphic site.
